(19) United States
(12) Patent Application Publication
(10) Pub. No.: US 2022/0330978 A1
(43) Pub. Date: Oct. 20, 2022

(54) STIMULATION ASSEMBLY SHEATH WITH SIGNAL PATHWAY

(71) Applicant: Cochlear Limited, Macquarie University, NSW (AU)

(72) Inventors: Zachary Mark Smith, Pymble (AU); Nicholas Charles Kendall Pawsey, North Ryde (AU)

(21) Appl. No.: 17/754,085

(22) PCT Filed: Oct. 9, 2020
(86) PCT No.: PCT/IB2020/059530

§ 371 Date: Mar. 23, 2022

Related U.S. Application Data
(60) Provisional application No. 62/914,726, filed on Oct. 14, 2019.

(51) Int. Cl. A61B 17/34; A61N 1/05; A61N 1/36

(52) U.S. Cl. CPC A61B 17/3468; A61N 1/0541; A61N 1/36038

(57) ABSTRACT

An apparatus includes a cannula with at least one wall portion having an electrically insulating material. The at least one wall portion at least partially bounds a region configured to contain a portion of a medical implant system configured to be implanted on or within a recipient. The cannula further includes at least one portion configured to provide at least one electrically conductive pathway through the at least one wall portion from within the cannula to a region outside the cannula.

### BACKGROUND

### Field

The present application relates generally to implanted medical systems, and more specifically systems and methods for facilitating positioning of stimulation elements of such medical systems during implantation.

### Description of the Related Art

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In one aspect disclosed herein, an apparatus comprises a cannula that comprises at least one wall portion comprising an electrically insulating material. The at least one wall portion at least partially bounds a region configured to contain a portion of a medical implant system configured to be implanted on or within a recipient. The cannula further comprises at least one portion configured to provide at least one electrically conductive pathway through the at least one wall portion from within the cannula to a region outside the cannula.

In another aspect disclosed herein, an apparatus comprises a body configured to contain a portion of a medical implant system configured to be implanted on or within a recipient. The apparatus further comprises at least one channel within a wall of the body and extending along the wall to an end portion of the body. The at least one channel is configured to receive fluid configured to provide electrical conductivity from the portion of the medical implant system to a region outside the body.

In yet another aspect disclosed herein, a method comprises providing a stimulation assembly of a medical implant system configured to be implanted on or within a recipient. The stimulation assembly is at least partially contained within a first region within an insertion sheath comprising an electrically insulating material between the first region and a second region outside the insertion sheath. The method further comprises, using the stimulation assembly to perform at least one electrical measurement indicative of the second region while the insertion sheath is at least partially inserted into a portion of a body of a recipient.

### BRIEF DESCRIPTION OF THE DRAWINGS

Embodiments are described herein in conjunction with the accompanying drawings, in which:

FIG. 1 is a perspective view of an example cochlear implant auditory prosthesis implanted in a recipient in accordance with certain embodiments described herein;

FIGS. 2A-2F schematically illustrate various configurations during implantation of a perimodiolar stimulation assembly into the cochlea of the recipient in accordance with certain embodiments described herein;

FIGS. 3A-3D schematically illustrate perspective views of various example apparatus in accordance with certain embodiments described herein;

FIG. 4A schematically illustrates a side view of another example apparatus in accordance with certain embodiments described herein;

FIG. 4B schematically illustrates a cross-sectional view of a portion of the example apparatus of FIG. 4A with the region containing a stimulation assembly in accordance with certain embodiments described herein;

FIG. 5 schematically illustrates a cross-sectional view of a portion of an example apparatus with the at least one electrically conductive material extending across multiple orifices in accordance with certain embodiments described herein;

FIG. 6 schematically illustrates a cross-sectional view of another example apparatus in accordance with certain embodiments described herein; and

FIG. 7 is a flow diagram of an example method in accordance with certain embodiments described herein.

### DETAILED DESCRIPTION

A medical device (e.g., a cochlear implant auditory prosthesis system) can include an elongate stimulation assembly (e.g., electrode array) at least partially enveloped by an insertion sheath configured to protect the stimulation assembly during the implantation process and/or to constrain a pre-curved stimulation assembly to be in a substantially straight configuration during the implantation process. However, until the insertion sheath is removed, the insertion sheath prevents the stimulation elements (e.g., electrodes) of the stimulation assembly from making electrical contact with the portions of the recipient's body (e.g., cochlear fluid), thereby preventing electrical measurements indicative of an environment containing the stimulation assembly (e.g., electrical voltage measurements; electrocochleography measurements; impedance measurements) during the implantation process (e.g., during insertion of the stimulation assembly into the recipient's body) which could otherwise be used in real-time feedback techniques to monitor and/or facilitate the implantation process.

Certain embodiments described herein provide an insertion sheath which includes one or more passive features (e.g., holes; slots; electrically conductive portions) which provide one or more electrically conductive pathways from the stimulation elements of the stimulation assembly within the insertion sheath to the surrounding environment of the recipient's body during the implantation process (e.g., during insertion of the stimulation assembly into the recipient's body). The insertion sheath of certain such embodiments can advantageously enable real-time electrical voltage measurements using the stimulation elements during the implantation process.

The teachings detailed herein are applicable, in at least some embodiments, to any type of implantable medical device (e.g., implantable sensory prostheses) configured to provide stimulation signals to the recipient of the implantable medical device. For example, the implantable medical device can comprise an auditory prosthesis system utilizing an implantable actuator assembly that generates electrical and/or optical stimulation signals to the recipient that are perceived by the recipient as sounds. Examples of auditory prosthesis systems compatible with certain embodiments described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Embodiments can include any type of medical device that can utilize the teachings detailed herein and/or variations thereof. In some embodiments, the teachings detailed herein and/or variations thereof can be utilized in other types of implantable medical devices beyond auditory prostheses. For example, the concepts described herein can be applied to any of a variety of implantable medical devices comprising an implanted component configured to provide stimulation signals (e.g., electrical, optical, and/or other stimulation signals) to the recipient of the implanted component so as to communicate information to the recipient of the implanted component. For example, such implantable medical devices can include one or more of the following: visual prostheses (e.g., retinal implants); brain implants; seizure devices (e.g., devices for monitoring and/or treating epileptic events); sleep apnea devices; functional electrical stimulation devices.

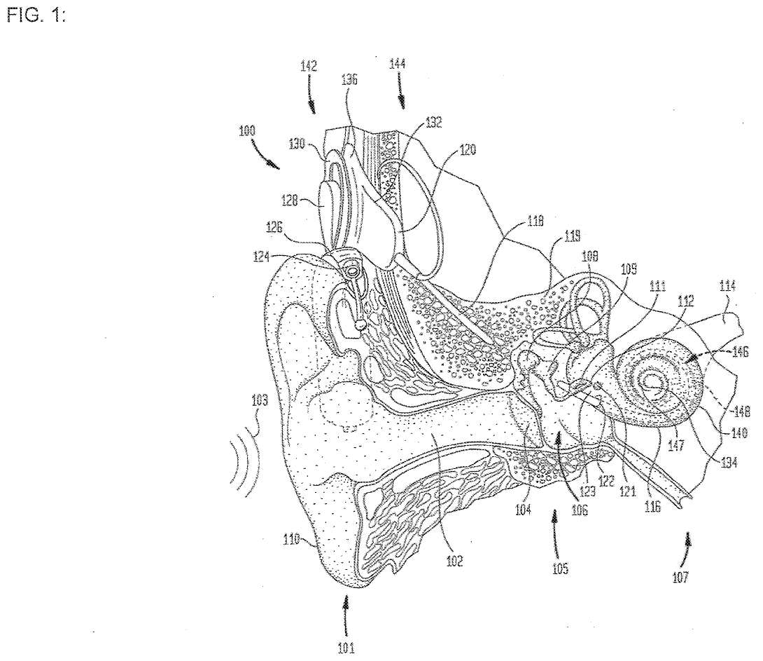

FIG. 1 is a perspective view of an example cochlear implant auditory prosthesis 100 implanted in a recipient in accordance with certain embodiments described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain embodiments described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other embodiments, the sound processing unit 126 has other arrangements, such as by an OTE processing unit (e.g., a component having a generally cylindrical shape and which is configured to be magnetically coupled to the recipient's head), etc., a mini or micro-BTE unit, an in-the-canal unit that is configured to be located in the recipient's ear canal, a body-worn sound processing unit, etc.

The sound processing unit 126 of certain embodiments includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative embodiments of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted embodiment of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some embodiments, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some embodiments, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other embodiments, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

In certain embodiments, the stimulation assembly 118 is contained within a protective sheath during at least a portion of the implantation process. A variety of types of stimulation assemblies 118 are compatible with certain embodiments described herein (e.g., straight; curved; elongated; short). In certain embodiments described herein, the stimulation assembly 118 is configured to provide information regarding the position of the stimulation assembly 118 during implantation into the recipient's body. For example, the one or more stimulation elements 148 of the stimulation assembly 118 can be configured to provide information regarding the position of the stimulation assembly 118 during implantation into the recipient's body and to provide the stimulation signals to the recipient's body during operation of the medical device after implantation is completed (e.g., to directly stimulate cells within the cochlea 140 to create nerve impulses resulting in perception of a received sound by the recipient (e.g., to evoke a hearing precept). For another example, the stimulation assembly 118 can comprise one or more sensors, distinct from the stimulation elements 148 of the stimulation assembly 118, that provide information regarding the position of the stimulation assembly 118 during implantation into the recipient's body.

In certain embodiments, a perimodiolar stimulation assembly 118 is configured to adopt a curved configuration during and/or after implantation into the cochlea 140. To achieve this, in certain embodiments, the perimodiolar stimulation assembly 118 is pre-curved to the same general curvature of the cochlea 140 but is kept in a straight configuration during at least a portion of the implantation process. For example, some perimodiolar stimulation assemblies 118 comprise varying material combinations or the use of shape memory materials, so that the stimulation assembly 118 may adopt its curved configuration when in the cochlea 140. Other example perimodiolar stimulation assemblies 118 can be constrained (e.g., held) straight by, for example, a stiffening stylet (e.g., straight rod) contained within the stimulation assembly 118 and is removed from the stimulation assembly 118 during implantation. In certain other embodiments, the protective sheath which contains the stimulation assembly 118 is configured to constrain (e.g., hold) the stimulation assembly 118 in a substantially straight configuration and is configured to be removed from the stimulation assembly 118 during the implantation process.

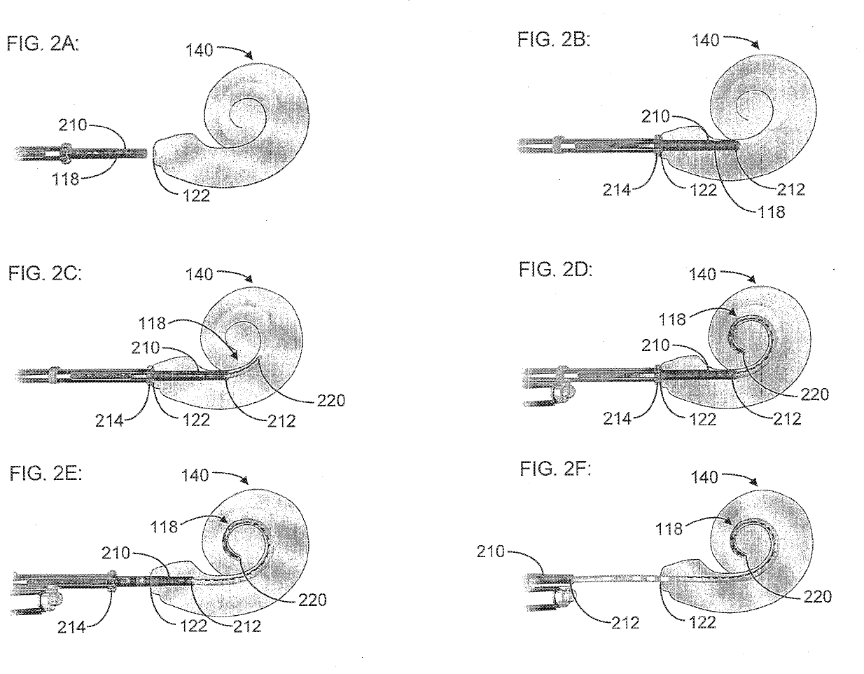

FIGS. 2A-2F schematically illustrate various configurations during implantation of a perimodiolar stimulation assembly 118 into the cochlea 140 of the recipient in accordance with certain embodiments described herein. The perimodiolar stimulation assembly 118 is substantially enclosed in a sheath 210 (e.g., cannula) configured to protect the stimulation assembly 118 during the implantation process. The sheath 210 is further configured to provide sufficient rigidity to maintain the pre-curved perimodiolar stimulation assembly 118 in a substantially straight configuration during at least a portion of the implantation process.

In certain embodiments, the implantation process includes creating an opening (e.g., facial recess) through the recipient's mastoid bone 119 (see, e.g., FIG. 1) to access the recipient's middle ear cavity 106 (see, e.g., FIG. 1). A cochleostomy 122 is created from the middle ear 106 into the cochlea 140 (e.g., through the round window 121, oval window 112, the promontory 123, etc. of the cochlea 140). The stimulation assembly 118 and the surrounding sheath 210 are advanced (e.g., pushed) through the opening through the mastoid bone 119 and are positioned to be inserted into the cochleostomy 122, as schematically illustrated in FIG. 2A.

As schematically illustrated in FIG. 2B, the stimulation assembly 118 and the sheath 210 are advanced (e.g., pushed) together through the cochleostomy 122 to insert a distal end portion 212 of the sheath 210 within the cochlea 140 while the stimulation assembly 118 remains in the sheath 210. The advancement of the stimulation assembly 118 and the surrounding sheath 210 is stopped once a proximal end portion 214 of the sheath 210 is in a predetermined position (e.g., in contact with the cochlea 140 at the cochleostomy 122). As schematically illustrated by FIG. 2C, the stimulation assembly 118 is then gently advanced (e.g., pushed) forward into the cochlea 140 through the sheath 210 (e.g., a distal end portion 220 of the stimulation assembly 118 exits the sheath 210 through an opening in the distal end portion 212 of the sheath 210). The portion of the stimulation assembly 118 that extends out of the sheath 210 is no longer constrained to be straight by the stimulation assembly 118 and therefore returns to its pre-curved configuration to follow the curvature of the canals within the cochlea 140. As schematically illustrated by FIG. 2D, the advancement of the stimulation assembly 118 continues until the stimulation assembly 118 achieves the implanted position (e.g., the distal end portion 220 of the stimulation assembly 118 is at the cochlea apex 134. For example, the implanted position can be the position at which the distal end portion 220 of the stimulation assembly 118 is placed at a selected angular position (e.g., angular insertion depth; angular rotation of the distal end portion 220 of the stimulation assembly 118 from the cochleostomy 122 through which the stimulation assembly 118 enters the cochlea 140). Once the stimulation assembly 118 achieves the implanted position, the sheath 200 can be withdrawn from the cochlea 140 (e.g., pulled out) through the cochleostomy 122, as schematically illustrated by FIGS. 2E-2F.

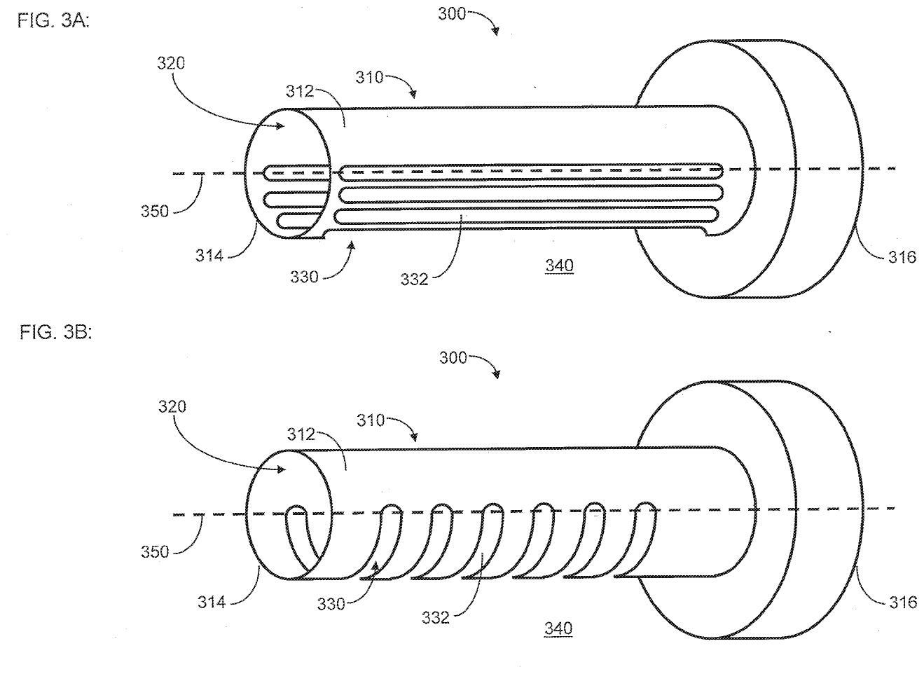

FIGS. 3A-3D schematically illustrate perspective views of various example apparatus 300 (e.g., insertion sheath) in accordance with certain embodiments described herein. The apparatus 300 comprises a cannula 310 comprising at least one wall portion 312 comprising an electrically insulating material. The at least one wall portion 312 at least partially bounds a region 320 configured to contain a portion of a medical implant system (e.g., a stimulation assembly 118; not shown in FIGS. 3A-3D) configured to be implanted on or within a recipient. The apparatus 300 further comprises at least one portion 330 configured to provide at least one electrically conductive pathway through the at least one wall portion 312 from within the cannula 310 to a region 340 outside the cannula 310.

In certain embodiments, the medical implant system can comprise a cochlear implant system and the portion of the medical implant system can comprise a stimulation assembly 118 (e.g., having an array 146 of stimulation elements 148; an array 146 of electrodes 148) configured to be implanted within a cochlea 140 of the recipient. The apparatus 300 of certain embodiments is configured to facilitate implantation of the array 146 into the cochlea 140 (see, e.g., FIGS. 2A-2F).

In certain embodiments, the cannula 310 comprises a distal end portion 314, a proximal end portion 316, and has a length L in a range of 4 millimeters to 5 millimeters from the distal end portion 314 to the proximal end portion 316. Other lengths are also compatible with certain embodiments described herein. In certain embodiments, the at least one wall portion 312 is substantially cylindrical with a substantially circular cross-section in a plane perpendicular to the longitudinal axis 350 of the cannula 310. Other shapes and cross-sections are also compatible with certain embodiments described herein. In certain embodiments, the electrically insulating material of the at least one wall portion 312 comprises a non-conductive polymer (e.g., polyimide). Other electrically insulating materials are also compatible with certain embodiments described herein.

In certain embodiments, the portion of the medical implant system is flexible and has a pre-curved bias, and the cannula 310 is configured to restrain the flexible portion to have a substantially straight configuration when the flexible portion is within the region 320 and to allow the flexible portion to move through an end portion of the cannula 310 and to have a substantially curved configuration. In certain such embodiments, the at least one wall portion 312 has a stiffness (e.g., rigidity) larger than a stiffness of the flexible portion, such that the flexible portion within the region 320 is held in the substantially straight configuration and the flexible portion outside the region 320 is in the substantially curved configuration. For example, the portion of the medical implant system can comprise a perimodiolar stimulation assembly 118 having a flexible electrode array 146, and the cannula 310 can be configured to restrain the electrode array 146 to have a substantially straight configuration when the electrode array 146 is within the region 320 and to allow the electrode array 146 to move through the distal end portion 314 of the cannula 310 and to have a substantially curved configuration within the cochlea 140 of the recipient.

As schematically illustrated by FIGS. 3A-3D, in certain embodiments, the at least one portion 330 comprises at least one orifice 332 through the at least one wall portion 312. The at least one orifice 332 is configured to, during implantation of the portion of the medical implant system on or within the recipient, allow fluid (e.g., from the recipient) to flow into the at least one orifice 322, the fluid providing the at least one electrically conductive pathway. In certain embodiments, the at least one orifice 332 has a size sufficient for the recipient's bodily fluid to flow into the at least one orifice 332 (e.g., through the orifice 332 and into the cannula 310), creating at least one electrical pathway through the bodily fluid from the portion of the medical implant system within the cannula 310 to the region 340 outside the cannula 310. For example, the at least one orifice 332 can have a size sufficient for the recipient's cochlear fluid to flow into the at least one orifice 332, making electrical contact with the electrode array 146 of the stimulation assembly 118 within the region 320 and creating electrical pathways through the cochlear fluid from the electrode array 146 to the region 340 outside the cannula 310.

In certain other embodiments, the region 320 containing the portion of the medical implant system also contains a biocompatible and electrically conductive fluid (e.g., saline). For example, the apparatus 300 can contain the portion of the medical implant system and the fluid prior to being inserted into the recipient's body (e.g., the apparatus 300 can be “pre-charged” with the fluid) and the at least one orifice 332 has a size sufficient for the recipient's bodily fluid and/or the pre-charged fluid to flow into the at least one orifice 332, creating the at least one electrical pathway via the bodily fluid and/or the pre-charged fluid.

As schematically illustrated by FIG. 3A, in certain embodiments, the at least one orifice 332 comprises at least one elongate slot extending in a direction substantially parallel to the longitudinal axis 350 of the cannula 310. The at least one slot can have a width in a direction around the longitudinal axis 350 that is less than or equal to a width of the stimulation elements 148 (e.g., electrodes) of the portion of the medical implant system within the cannula 310 but that is sufficiently large to allow the recipient's bodily fluid and/or pre-charged fluid to flow therein. The at least one slot can have a length in a direction substantially parallel to the longitudinal axis 350 such that the stimulation elements 148 within the cannula 310 are continually in electrical communication with the bodily fluid in the region 340 outside the cannula 310 while the portion of the medical implant system (e.g., the stimulation assembly 118) is advanced through the cannula 310.

As schematically illustrated by FIG. 3B, in certain embodiments, the at least one orifice 332 comprises at least one elongate slot extending in a direction around the longitudinal axis 350 of the cannula 310. The at least one slot can have a width in a direction substantially parallel to the longitudinal axis 350 that is less than or equal to a width of the stimulation elements 148 (e.g., electrodes) of the portion of the medical implant system within the cannula 310 but that is sufficiently large to allow the recipient's bodily fluid and/or pre-charged fluid to flow therein. The at least one slot can have a length in a direction around the longitudinal axis 350 such that the stimulation elements 148 within the cannula 310 are in electrical communication with the bodily fluid in the region 340 outside the cannula 310 for a range of orientations of the stimulation elements 148 within the cannula 310 (e.g., in a range of angles about the longitudinal axis 350).

While both FIGS. 3A-3B schematically illustrate a plurality of slots, in certain other embodiments, the apparatus 300 comprises only a single slot. While both FIGS. 3A-3B schematically illustrate a plurality of slots that are substantially parallel to one another, in certain other embodiments, the slots are not substantially parallel to one another (e.g., at a non-zero angle relative to one another). While both FIGS. 3A-3B schematically illustrate a plurality of slots that are substantially straight, in certain other embodiments, the slots are not substantially straight (e.g., curved; serpentine-shaped).

As schematically illustrated by FIGS. 3C-3D, in certain embodiments, the at least one orifice 332 comprises a plurality of holes. In certain embodiments, the holes are distributed in a periodic pattern (e.g., as schematically illustrated by FIGS. 3C-3D), while in certain other embodiments, the holes are distributed in a non-periodic pattern (e.g., random pattern). While FIG. 3A schematically illustrates a plurality of substantially circular holes in a rectilinear pattern and FIG. 3B schematically illustrates a mesh having a plurality of substantially rectangular holes in a rectilinear pattern, other shapes and patterns of the holes are also compatible with certain embodiments described herein.

In certain embodiments, each of the holes has a size (e.g., width; length) that is less than or equal to a width of the stimulation elements 148 (e.g., electrodes) of the portion of the medical implant system within the cannula 310 but that is sufficiently large to allow the recipient's bodily fluid and/or pre-charged fluid to flow therein. For example, the holes can be sized and distributed such that each stimulation element 148 within the cannula 310 is aligned (e.g., registered) with two or more holes (e.g., four holes per stimulation element 148) such that the two or more holes provide an electrically conductive pathway from the stimulation element 148 to the region 340 outside the cannula 310.

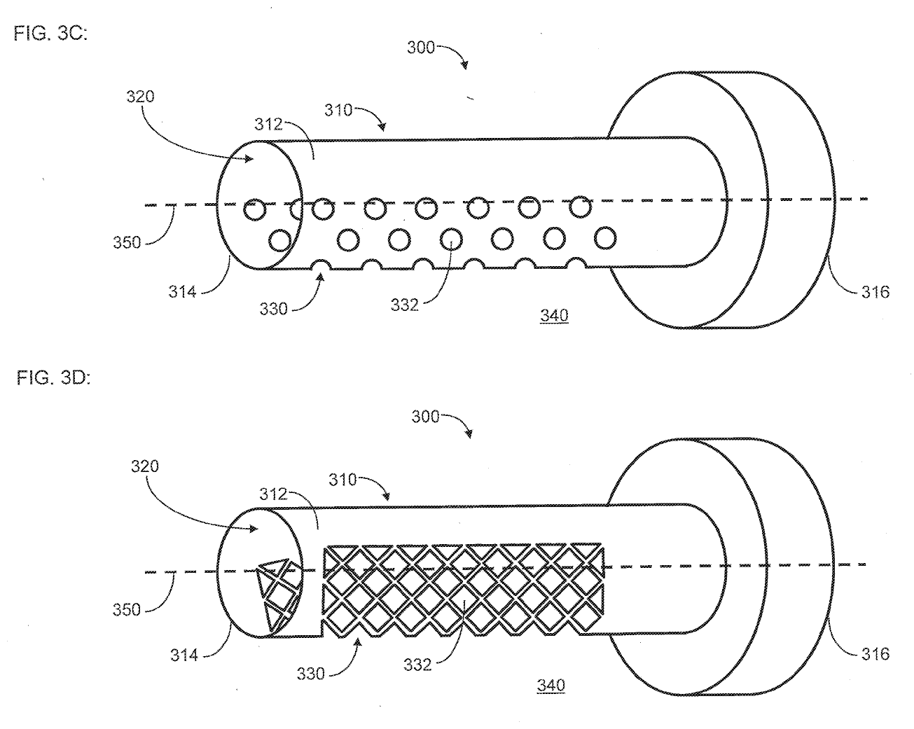

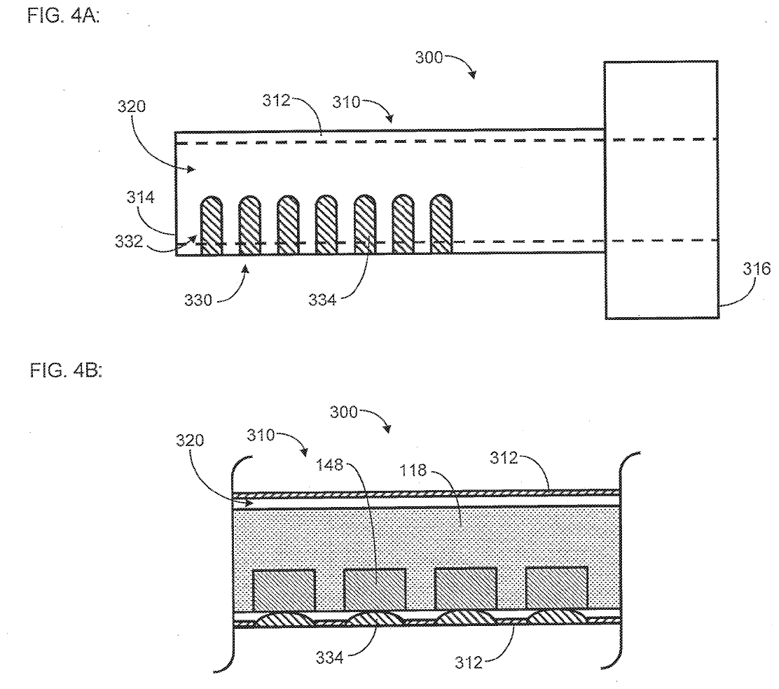

FIG. 4A schematically illustrates a side view of another example apparatus 300 (e.g., insertion sheath) in accordance with certain embodiments described herein. FIG. 4B schematically illustrates a cross-sectional view of a portion of the example apparatus 300 of FIG. 4A with the region 320 containing a stimulation assembly 118 in accordance with certain embodiments described herein. The at least one portion 330 comprises at least one electrically conductive solid material 334 extending through the at least one wall portion 312 and providing the at least one electrically conductive pathway. Examples of the at least one electrically conductive solid material 334 include, but are not limited to: biocompatible and electrically conductive elastomer; electrically conductive silicone. In certain embodiments, the at least one electrically conductive solid material 334 has an electrical resistance and/or electrical impedance that is substantially equal to that of the recipient's bodily fluid (e.g., cochlear fluid) in which the apparatus 300 is to be inserted.

In certain embodiments, the at least one portion 330 comprises at least one orifice 332 extending through the at least one wall portion 312 and the at least one electrically conductive solid material 334 is within (e.g., fills) the at least one orifice 332. For example, as schematically illustrated by FIG. 4A, the at least one orifice 332 comprises a plurality of slots (see, e.g., FIG. 3B) and the slots contain the at least one electrically conductive solid material 334. In certain other embodiments, other shapes and configurations of the at least one orifice 332 as described herein (see, e.g., FIGS. 3A, 3C, and 3D) contain the at least one electrically conductive solid material 334. While FIG. 4B schematically illustrates an example embodiment in which the at least one electrically conductive solid material 334 form bumps within the region 320, in certain other embodiments, the at least one electrically conductive solid material 334 is substantially flat within the region 320. While FIG. 4B schematically illustrates an example embodiment in which the at least one electrically conductive solid material 334 is in contact with the stimulation elements 148 (e.g., electrodes) of the stimulation assembly 118, in certain other embodiments, the at least one electrically conductive solid material 334 is spaced from the stimulation elements 148 and an electrically conductive fluid (e.g., saline) within the region 320 is in electrical communication with the stimulation elements 148 and the at least one electrically conductive solid material 334.

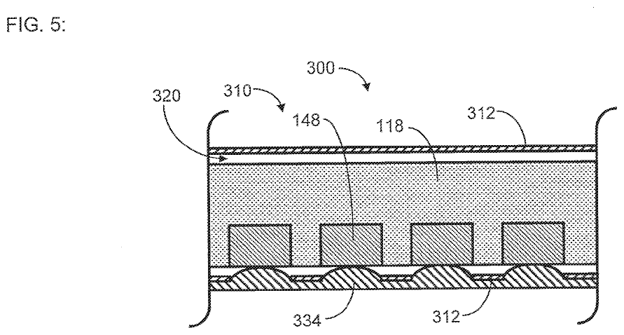

In certain embodiments, as schematically illustrated by FIGS. 4A-4B, the at least one electrically conductive solid material 334 in the different orifices 332 are electrically isolated from one another (e.g., spaced by the electrically insulating at least one wall portion 312). In certain other embodiments, as schematically illustrated by FIG. 5, the at least one electrically conductive solid material 334 extends across multiple orifices 332 (e.g., over an outer surface of the at least one wall portion 312). In certain such embodiments, the at least one electrically conductive solid material 334 has an electrical resistance and/or electrical impedance that is substantially equal to that of the recipient's bodily fluid (e.g., cochlear fluid) in which the apparatus 300 is to be inserted.

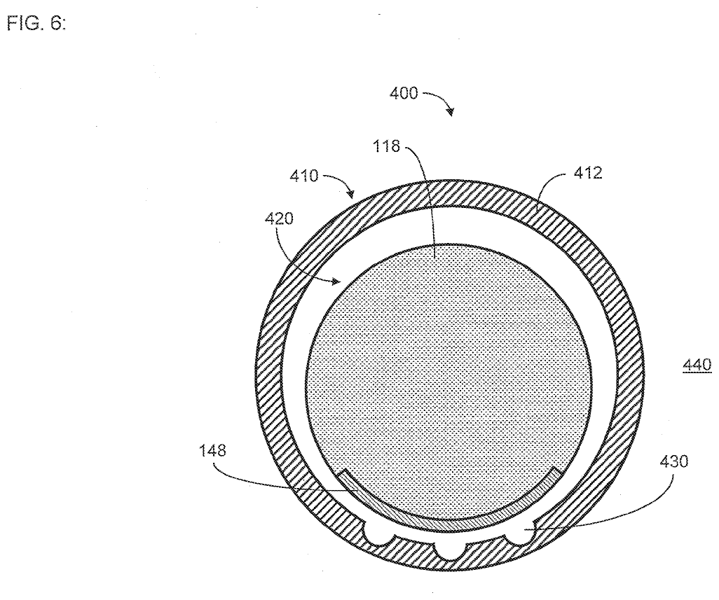

FIG. 6 schematically illustrates a cross-sectional view of another example apparatus 400 (e.g., insertion sheath) in accordance with certain embodiments described herein. The apparatus 400 comprises a body 410 (e.g., a cannula 310 at least partially bounding a region 420) configured to contain a portion of a medical implant system (e.g., a stimulation assembly 118 of a cochlear implant system within the region 420) configured to be implanted on or within a recipient. The apparatus 400 further comprises at least one channel 430 within a wall 412 of the body 410 and extending along the wall 412 to an end portion of the body 410. For example, the at least one channel 430 can extend along a longitudinal axis 350 of the cannula 310 to a distal end portion 314 of the cannula 310. The at least one channel 430 is configured to receive fluid (e.g., saline and/or recipient's bodily fluid) configured to provide electrical conductivity from the portion of the medical implant system to a region 440 outside the body 410. For example, saline and/or recipient's cochlear fluid can provide electrical conductivity from the stimulation elements 148 of the stimulation assembly 118 in the region 420 to the region 440 outside the body 410.

In certain embodiments, the at least one channel 430 extends a length in a direction substantially parallel to the longitudinal axis 350 such that the stimulation elements 148 within the region 420 are continually in electrical communication with the fluid in the at least one channel 430 while the portion of the medical implant system (e.g., the stimulation assembly 118) is advanced through the body 410. While FIG. 6 schematically illustrates a plurality of channels 430, in certain other embodiments, the body 410 comprises a single channel. In certain embodiments having a plurality of channels 430, the channels 430 are substantially parallel to one another, while in certain other embodiments, the channels 430 are not substantially parallel to one another (e.g., at a non-zero angle relative to one another). In certain embodiments, the at least one channel 430 is substantially straight, while in certain other embodiments, the at least one channel 430 is not substantially straight (e.g., curved; serpentine-shaped). In certain embodiments, as schematically illustrated by FIG. 6, the at least one channel 430 has a curved shape in a plane substantially perpendicular to the longitudinal axis 350 of the body 410 (e.g., cannula 310). In certain other embodiments, the at least one channel 430 has another shape (e.g., rectangular; geometric; non-geometric) in a plane substantially perpendicular to the longitudinal axis 350. In certain embodiments, the width of the at least one channel 430 is configured to allow the fluid to flow along the at least one channel 430, while in certain other embodiments, the width of the at least one channel 430 is configured to retain the fluid (e.g., via surface tension) within the at least one channel 430.

In certain embodiments, the body 410 (e.g., cannula 310) comprises an electrically insulating material which comprises a non-conductive polymer (e.g., polyimide) and/or one or more other electrically insulating materials. In certain embodiments, the fluid is within the at least one channel 430 prior to implantation of the portion of the medical implant system on or within the recipient (e.g., saline pre-charged within the region 420 with the stimulation assembly 118). In certain other embodiments, the fluid comprises a bodily fluid of the recipient and is within the at least one channel 430 during and after implantation of the portion of the medical implant system on or within the recipient.

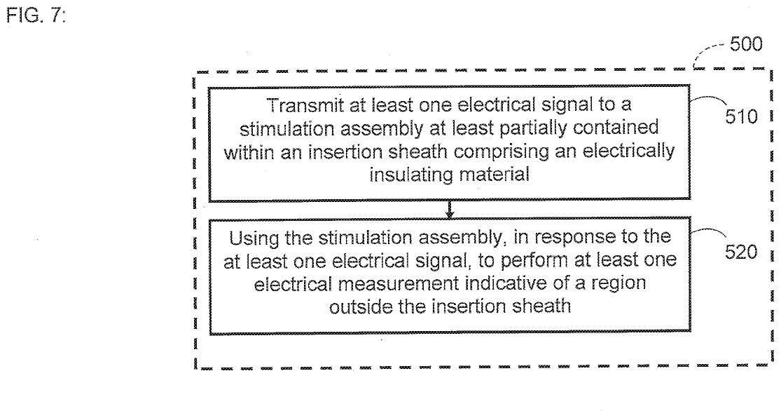

FIG. 7 is a flow diagram of an example method 500 in accordance with certain embodiments described herein. While the method 500 is described herein with reference to the structures of FIGS. 3A-3D, 4A-4B, 5, and 6, the method 500 is compatible with other structures as well.

In an operational block 510, the method 500 comprises providing a stimulation assembly of a medical implant system configured to be implanted on or within a recipient. For example, the stimulation assembly can comprise a plurality of stimulation elements 148 (e.g., electrodes) of a cochlear implant auditory prosthesis system 100, the stimulation elements 148 in electrical communication with at least one other portion of the auditory prosthesis system 100 (e.g., the stimulator unit 120 of the internal component 144). The stimulation assembly is at least partially contained in a first region 320, 420 within a cannula 310 or body 410 (e.g., insertion sheath) comprising an electrically insulating material (e.g., a non-conductive polymer; polyimide) between the first region 320, 420 and a second region 340, 440 outside the cannula 310 or body 410. For example, at least some of the stimulation elements 148 are contained within the first region 320, 420.

In an operational block 520, the method 500 further comprises using the stimulation assembly to perform at least one electrical measurement indicative of the second region 340 while the cannula 310 or body 410 is at least partially inserted into a portion of a body of a recipient (e.g., a cochlea 140 of the recipient). In certain embodiments, the at least one electrical measurement comprises using at least one of the stimulation elements 148 within the cannula 310 to receive electrical signals from the second region 340 in response to acoustic signals transmitted to the cochlea 140 (e.g., at least one electrocochleography measurement). In certain other embodiments, the at least one electrical measurement comprises using at least one of the stimulation elements 148 within the cannula 310 to transmit electrical signals to the second region 340 and/or using at least one of the stimulation elements 148 within the cannula 310 to receive electrical signals from the second region 340 (e.g., at least one transimpedance measurement; at least one electrical voltage measurement; at least one impedance measurement). For example, a first stimulation element 148a can be used to transmit an first electrical signal to the second region 340 and a second stimulation element 148b can be used to receive a second electrical signal from the second region 340. The first stimulation element 148a can be inside the cannula 310 and the second stimulation element 148b can be outside the cannula 310, the first stimulation element 148a can be outside the cannula 310 and the second stimulation element 148b can be inside the cannula 310, or both the first stimulation element 148a and the second stimulation element 148b can be inside the cannula 310.

In certain embodiments, the at least one electrical measurement is performed via at least one electrically conductive pathway through at least one orifice 330 extending through a wall portion 312, 412 of the cannula 310 or body 410. For example, the at least one orifice 330 can be filled with a bodily fluid of the recipient (e.g., cochlear fluid). For another example, the at least one orifice 330 can be filled with an electrically conductive fluid (e.g., saline). In certain embodiments, the at least one orifice 330 is filed with an electrically conductive solid material (e.g., electrically conductive silicone).

In certain embodiments, the method 500 further comprises using the at least one electrical measurement to generate information indicative of implantation of the stimulation assembly on or within the recipient while the implantation is being performed. For example, the at least one electrical measurement can be used to generate real-time information indicative of the implantation of the stimulation assembly of the cochlear implant auditory prosthesis system into the recipient's cochlea while the implantation is being performed. Such real-time information can be provided to the health practitioner (e.g., surgeon) and/or to an computer-controlled (e.g., robotic) insertion system performing the implantation (e.g., to be used as feedback information as the stimulation assembly is being inserted).

It is to be appreciated that the embodiments disclosed herein are not mutually exclusive and may be combined with one another in various arrangements. In addition, although the disclosed methods and apparatuses have largely been described in the context of conventional cochlear implants, various embodiments described herein can be incorporated in a variety of other suitable devices, methods, and contexts. More generally, as can be appreciated, certain embodiments described herein can be used in a variety of implantable medical device contexts that can benefit from a signal pathway between the stimulation assembly and the recipient during implantation (e.g., insertion) of the stimulation assembly.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree.

The invention described and claimed herein is not to be limited in scope by the specific example embodiments herein disclosed, since these embodiments are intended as illustrations, and not limitations, of several aspects of the invention. Any equivalent embodiments are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example embodiments disclosed herein, but should be defined only in accordance with the claims and their equivalents.

1. An apparatus comprising:
a cannula comprising:
at least one wall portion comprising an electrically insulating material, the at least one wall portion at least partially bounding a region configured to contain a portion of a medical implant system configured to be implanted on or within a recipient; and
at least one portion configured to provide at least one electrically conductive pathway through the at least one wall portion from within the cannula to a region outside the cannula.

2. The apparatus of claim 1, wherein the at least one portion comprises at least one orifice through the at least one wall portion, the at least one orifice configured to, during implantation of the portion of the medical implant system on or within the recipient, allow fluid to flow into the at least one orifice, the fluid providing the at least one electrically conductive pathway.

3. The apparatus of claim 2, wherein the at least one orifice comprises at least one slot extending in a direction substantially parallel to a longitudinal axis of the cannula.

4. The apparatus of claim 2, wherein the at least one orifice comprises at least one slot extending in a direction around a longitudinal axis of the cannula.

5. The apparatus of claim 1, wherein the at least one orifice comprises a plurality of slots that are substantially parallel to one another.

6. The apparatus of claim 5, wherein the slots are substantially straight.

7. The apparatus of claim 5, wherein the slots are serpentine-shaped.

8. The apparatus of claim 1, wherein the at least one wall portion comprises a mesh and at least one orifice comprises a plurality of holes of the mesh.

9. The apparatus of claim 1, wherein the at least one portion comprises at least one electrically conductive material extending through the at least one wall portion, the at least one electrically conductive material providing the at least one electrically conductive pathway.

10. The apparatus of claim 1, wherein the medical implant system comprises a cochlear implant system and the portion comprises a stimulation assembly having an electrode array configured to be implanted within a cochlea of the recipient.

11. The apparatus of claim 10, wherein the electrode array is flexible and has a pre-curved bias, the cannula configured to restrain the electrode array to have a substantially straight configuration when the electrode array is within the region and to allow the electrode array to move into the cochlea through an end portion of the cannula and to have a substantially curved configuration.

12. An apparatus comprising:
a body configured to contain a portion of a medical implant system configured to be implanted on or within a recipient; and
at least one channel within a wall of the body and extending along the wall to an end portion of the body, the at least one channel configured to receive fluid configured to provide electrical conductivity from the portion of the medical implant system to a region outside the body.

13. The apparatus of claim 12, wherein the body comprises an electrically insulating material.

14. The apparatus of claim 12, wherein the fluid is within the at least one channel prior to implantation of the portion of the medical implant system on or within the recipient.

15. The apparatus of claim 12, wherein the fluid comprises a bodily fluid of the recipient and that is within the at least one channel during and after implantation of the portion of the medical implant system on or within the recipient.

16. A method comprising:
providing a stimulation assembly of a medical implant system configured to be implanted on or within a recipient, the stimulation assembly at least partially contained in a first region within an insertion sheath comprising an electrically insulating material between the first region and a second region outside the insertion sheath; and
using the stimulation assembly to perform at least one electrical measurement indicative of the second region while the insertion sheath is at least partially inserted into a portion of a body of a recipient.

17. The method of claim 16, further comprising using the at least one electrical measurement to generate information indicative of implantation of the stimulation assembly on or within the recipient while the implantation is being performed.

18. The method of claim 16, wherein the medical implant system comprises a cochlear implant auditory prosthesis system, the portion of the body of the recipient comprises a cochlea of the recipient, and the at least one electrical measurement is selected from the group consisting of: at least one transimpedance measurement; at least one electrical voltage measurement; at least one electrocochleography measurement; at least one impedance measurement.

19. The method of claim 16, wherein said at least one electrical measurement is performed via at least one electrically conductive pathway through at least one orifice extending through a wall portion of the insertion sheath.

20. The method of claim 19, wherein the at least one orifice is filled with a bodily fluid of the recipient.

21. The method of claim 19, wherein the at least one orifice is filled with an electrically conductive fluid.

22. The method of claim 19, wherein the at least one orifice is filled with an electrically conductive solid material.

23. The method of claim 16, wherein said at least one electrical measurement is performed via at least one electrically conductive pathway along at least one channel within a wall of the insertion sheath and extending along the wall to an end portion of the insertion sheath.
